(19) United States
(12) Patent Application Publication
(10) Pub. No.: US 2013/0062535 A1
(43) Pub. Date: Mar. 14, 2013

(54) SURFACE-PROCESSING DEVICE FOR A DENTAL IMPLANT

(71) Applicants: Kwang Bum Park, Daegu (KR); Dong Jun Yang, Daegu (KR); Hyun Wook An, Gyeongbuk (KR); Sun Young Lee, Daegu (KR); Chang Hee Yang, Busan (KR); Min Kim, Gwangju (KR); Byung Kwon Moon, Gwangju (KR); Hyun Seung Kim, Gwangju (KR); Chul Jung, Gwangju (KR); Chul Woong Jeong, Gwangju (KR)

(72) Inventors: Kwang Bum Park, Daegu (KR); Dong Jun Yang, Daegu (KR); Hyun Wook An, Gyeongbuk (KR); Sun Young Lee, Daegu (KR); Chang Hee Yang, Busan (KR); Min Kim, Gwangju (KR); Byung Kwon Moon, Gwangju (KR); Hyun Seung Kim, Gwangju (KR); Chul Jung, Gwangju (KR); Chul Woong Jeong, Gwangju (KR)

(73) Assignee: MEGAGEN IMPLANT CO. LTD., Gyeongbuk (KR)

(21) Appl. No.: 13/383,570

(22) PCT Filed: May 31, 2011
(86) PCT No.: PCT/KR2011/003987

§ 371 Date: Jan. 11, 2012

(30) Foreign Application Priority Data
May 31, 2010 (KR) 10-2010-0051036
May 29, 2011 (KR) 10-2011-0028367

(51) Int. Cl. A61L 2/00

(52) U.S. Cl. CPC A61L 2/00
USPC 250/455.11

(57) ABSTRACT

A surface treatment device of dental implant according to an embodiment of the present invention includes: a body providing a treatment chamber to receive a surface processing object; a cover combined to the body to selectively open the treatment chamber; a light source irradiating UV light to a surface of the surface treatment object; and a reflective plate positioned at a bottom of the treatment chamber to reflect the UV light emitting from the light source toward the surface treatment object.

### CLAIM OF PRIORITY

The present invention claims priority to Korean Patent Application 10-2010-0051036, filed on May 31, 2010, Korean Patent Application 10-2011-0028367, filed on Mar. 29, 2011, and to PCT/KR2011/003987 filed on May 31, 2011, all of which are assigned to MEGAGEN IMPLANT CO. LTD. and incorporated herein by reference.

### FIELD

The present invention relates to a surface treatment device of dental implant.

### BACKGROUND

People have interest in an oral health care depending on a progress of an aging society.

If tooth loss occurs, a masticatory function losses to cause a restriction of food intake and a digestive disorders and affect an interpersonal relationships due to a facial deformation and an incorrect pronunciation

In order to solve the mentioned-above problem, in the past, there was a prosthodontic treatment method using dentures and a bridges but it is growing fashionable that an implant treatment by which makes to be looked like a nature teeth as a cost for the implant treatment remarkably decreases. An implant treatment has been recognized as a universal treatment method for teeth loss as a consumer's income level increases and a dental technology develops.

A dental implant is used to permanently implant an artificial tooth on a human jaw and performs the same function as the nature tooth by connecting a jaw with artificial tooth and bearing and distributing a load generating when chewing the food. The dental implant is mechanically manufactured to serve as the more stable tooth. Therefore, the implant should be made of a very stable bio-compatible material for a human tissue. In addition, the implant can prevent a side effect of the body, other chemical or biochemical reactions and a human body rejection reaction. It is very difficult to select an appropriate material because the implant should have a high mechanical strength such that a deformation or destruction does not occur even under the action of the repetitive load and a momentary pressure.

An attempt has been made for development of various metals and alloys as an appropriate material of the implant, but currently, titanium metal or titanium alloy is mainly used. Titanium or titanium alloy has easy process ability, a high bio-compatibility for the human body tissue, a high mechanical strength and a bio-inert. However, it has disadvantages that titanium or titanium alloy takes a long time in an osseointegration when they are implanted into the human body and while time passes after the implant, metal ions melt into the human body.

In order to overcome these shortcomings, a technology which can enhance osseointegration has be developed and applied by appropriately handling surfaces of these materials. A speed and quality of the osseointegration have a close relationship with a surface property and a chemical composition such as a surface composition, a surface roughness and hydrophilic property. In particular, it has been reported that the implant having hydrophilic surface is preferable to adapt to an interactions with a biological fluid, a cell and tissue.

It has been reported that the implant coating the existing titanium surface with Titanium dioxide (TiO₂) allows the human body to be stable, provide an excellent biocompatibility and a positive side in view of a reaction with the cell and an oxide treated implant has more preferable osseointegration than that of a mechanically treated implant.

In order to coat the titanium oxide layer on the implant surface, the technology such as a plasma spraying, sputtering and an implantation is known.

However, an oxide film formed by the above-mentioned method doesn't have enough adhesive strength for a bone and has a disadvantage that process thereof is complicate.

In addition, more recently, the surface treated implant fixture is available as a product for a storage container for keeping the hydrophilic property for a long time. However, since fixture containing the storage container is expensiveness, it has a disadvantage that a consumer has a financial burden. In addition, the consumer should be used only for a particular company in spite of a high price. Therefore, it has a disadvantage that the consumer has a narrow choice for the product.

### SUMMARY

In order to improve the above-mentioned disadvantages, the object of the present invention is to provide an implant surface treatment device that can promote cell activation and syphilis by a simple process irradiating UV light on titanium oxide film formed in the surface of the implant.

In addition, the object of the present invention is to provide a dental implant surface treatment device that can prevent an external contamination or a bacterial infections by performing a high hydrophilic treatment on the implant surface just before the implant surgery and immediately implanting a patient.

In addition, an object of the present invention is to provide a surface treatment device that enables a commercially available product for all type of implants to be a general-purpose and provides a wide consumer choice and a low-cost.

In order to achieve the above-mentioned object, a surface treatment device of dental implant includes a body providing a treatment chamber to receive a surface processing object; a cover combined to the body to selectively open the treatment chamber; a light source irradiating UV light to a surface of the surface treatment object; and a reflective plate positioned at a bottom of the treatment chamber to reflect the UV light emitting from the light source toward the surface treatment object.

A dental surface treatment process device according to an embodiment of the present invention has the following effects.

First, in the present invention, there is an advantage that improves syphilis by reforming a titanium oxide with a hydrophilic property using a simple process irradiating a UV light on a surface of an implant.

Second, the present invention has a effect that may minimizes an infection phenomenon of an implanting site by performing a surface process just before an implanting surgery and implanting a surface treated implant into a patient to prevent an infection caused by an external containment or a bacterium.

Third, the present invention has an advantage that enables a commercially available product for all type of implants to be a general-purpose, provides a wide consumer choice and a low-cost implant minimizes a burden of a patient.

Fourth, the surface treatment can be performed on a plurality of implant member in one process, therefore surgical time for implanting a plurality of implant is reduced.

Fifth, since the UV light is irradiated in the implant while the implant member is rotated, there is an advantage that a uniform surface reforming is made across the entire implant surface.

### DESCRIPTION OF DRAWINGS

FIG. 1 is a cross-sectional view showing a treed state of a dental implant.

FIG. 2 is an exploded perspective view of a surface treatment device according to a first embodiment of the present invention.

FIG. 3 is a front view of the surface treatment device.

FIG. 4 is a cross-sectional view showing an implant member disposed at the surface treatment device.

FIG. 5 is a block diagram schematically showing a control system including a surface processing device of a dental implant according to a first embodiment of the present invention.

FIG. 6 is a graph analyzing a surface residue of a sample piece.

FIGS. 7 to 12 are a picture showing each of a droplet shape and an average contact angle formed at a control group and a test group.

FIG. 13 is a graph showing an absorbance of a control group and a test group.

FIGS. 14 to 18 is a picture showing an image of a cell enlarged at 250 magnifications, which the cell is attached to a control group and a test group.

FIGS. 19 to 23 is a picture showing an image of a cell enlarged at 1000 magnification, which the cell is attached to a control group and a test group.

FIG. 24 is a graph showing the number of quantified cells attached a control and a test group.

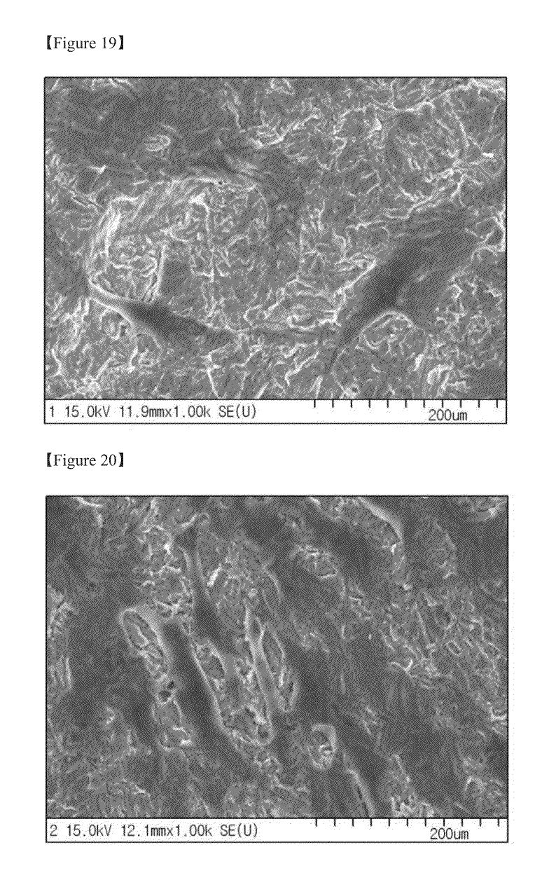

FIG. 25 is a front perspective view of a surface treatment device according to a second embodiment of the present invention.

FIG. 26 is a front perspective view showing an opened cover of the surface treatment processing device.

FIG. 27 is a rear perspective view showing an opened cover of the surface treatment processing device.

FIG. 28 is an exploded perspective view of the surface treatment device.

FIG. 29 is a longitudinal cross-sectional view taken along FIG. 25.

FIG. 30 is a block diagram schematically showing a control system including a surface processing device of a dental implant according to a second embodiment of the present invention.

### DESCRIPTION

A surface treatment device of a dental implant according to an embodiment of the present invention will be described with reference to drawings in detail.

Typically, a dental implant refers to a substitute for a lost natural teeth or to a dental surgery for restoring a original function of teeth by fastening a screw-shaped fixture to an alveolar bone and fusing with the bone during a predetermined period and then fixing a joining portion, for example an abutment and a prostheses such as artificial crown on the fixture fused with the bone The implant refers to a substitute for a lost natural tooth and in particular, to the fixture implanted into an alveolar periosteum. However, in addition to the fixture, it should be noted that the implant member combined with a cartilage cell is applied to the surface treatment by the surface treatment device according to an embodiment of the present invention.

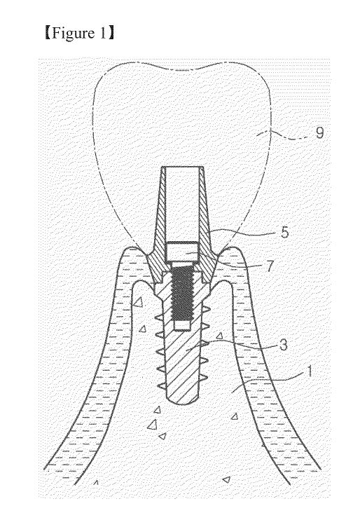

FIG. 1 is a cross-sectional view showing a treed state of a dental implant.

Referring to FIG. 1, the artificial tooth, for example substitute for the nature tooth, includes a fixture 3 inserted into alveolar periosteum, an abutment 5 combined to an upper portion of the fixture 3, a fixed screw 7 fixing the abutment 5 to the fixture 3 and an artificial crown 9 mounted over the abutment 5.

The present invention relates to a surface treatment device for increasing a hydrophilic property. The surface treatment device will be described with reference to drawings.

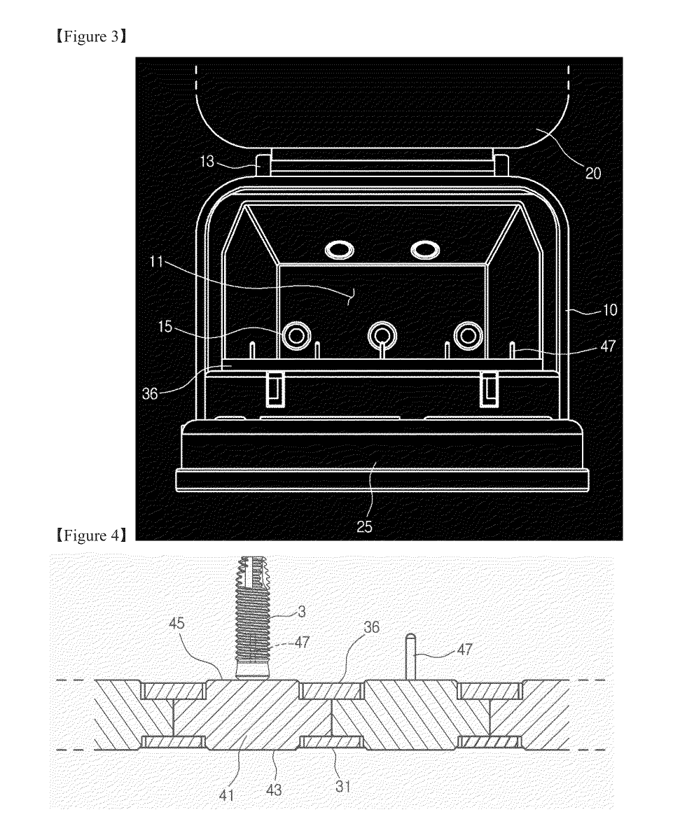

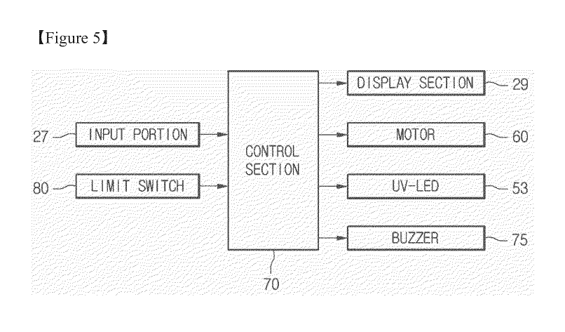

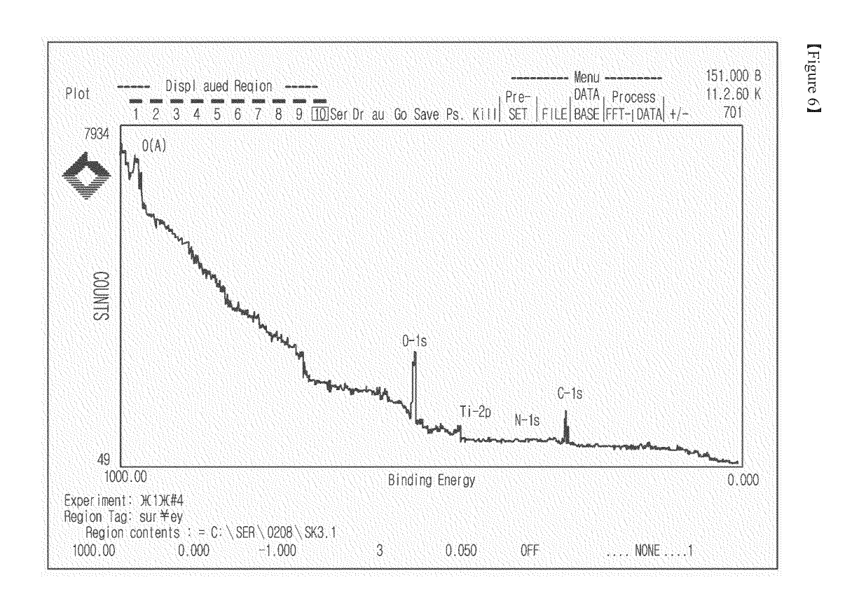

FIG. 3 is a front view of the surface treatment device. FIG. 4 is a cross-sectional view showing the surface treatment device disposed at the implant member and FIG. 5 is a block diagram schematically showing a control system including a surface processing device of a dental implant according to a first embodiment of the present invention.

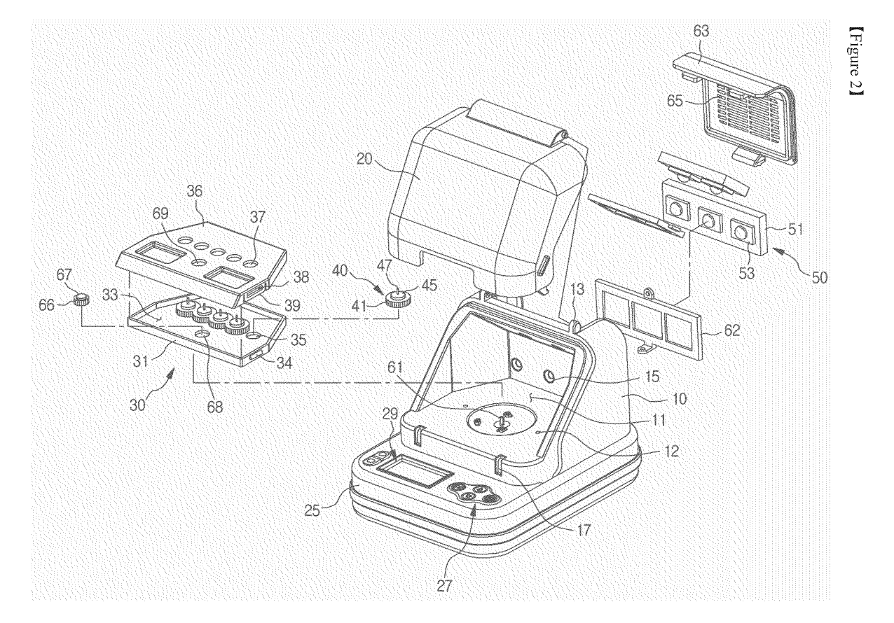

Referring to FIGS. 2 to 5, the surface treatment device according to an embodiment of the present invention includes a body 10, a door 20, a table 30, a light source array module 50 and a rotating means.

In more detail, a treatment chamber 11 for the treatment surface is formed within the body 10 and the front surface of the treatment chamber 11 has an opened structure. The door 20 is mounted at the opened front surface of the treatment chamber 11 to selectively open and close the treatment chamber 11. The door 20 is retractably connected upwardly and downwardly to the body 10 by a hinge combination to a bracket 13 formed at the upper portion of the body 10.

In addition, a base portion 25 supporting the body 10 is mounted at a lower portion of the body 10. The one side of the base portion 25 is provided with an input portion 27 for operating the surface treatment device and the other side thereof is provided with a display portion 29 for outputting much information. The input portion 27 includes a plurality of buttons capable of inputting commands (MENU, UP/STRAT, DN/PAUSE, SET) consisting of an output value and motors, the number of a motor rotation, an operating time and the like. In addition, an inner portion of the base portion 25 is mounted with a control portion and various electrical circuits.

In addition, the table 30 is mounted at a processing chamber 11 formed at the inner portion of the body 10. In detail, the table 30 is mounted to be fixed to the body or mounted to be separated from the body 10. An implant member is placed on the table 30.

The implant member 3 comprises the fixture coated with titanium at an outer periphery surface of an object made of titanium or made of other material except titanium. Titanium oxide film (TiO₂) is formed at the surface of the implant member 3. Titanium oxide film is in contact with air to be naturally or artificially formed on the surface of the implant member.

The surface processing device includes structure in which the implant member 3 attached to the table 30 is rotated by a rotating means. This can provide an irradiation of a uniform ultraviolet light on the surface of the implant member 3.

Meanwhile, the table 30 includes an lower cover 31, an upper cover 36 combined to the upper portion of the lower cover 31 and a plurality of rotating disk 40 retractably received to a receiving space 33 of the lower cover 36.

In detail, the receiving space 33 is formed within the lower cover 31 and the top portion of the lower cover is opened. In addition, the lower cover 31 is mounted to be separated from the body 10. A plurality of vertically protruding insert protrusions is formed at the bottom surface of the lower cover 31. The inserting protrusion is inserted into a combined hole 12 formed at the bottom of the body 10, specifically formed at the bottom of the processing chamber 11.

In addition, a plurality of first through-hole is formed at the lower cover 31 at predetermined interval. The first through-hole 35 has the same diameter, but may have different diameter as well.

The upper cover 36 is a structure having an opened lower portion, which is combined to the upper portion of the lower cover 31. In addition, the upper cover 36 may be detachable combined to the lower cover 31. To this end, an elastic piece 39 may be formed at both side surface of the upper surface 36. The one surface of elastic piece 30 integrally extend from the upper surface of the upper cover 36 and the remaining three surfaces thereof may chamfered from the side surface of the upper cover 36. The elastic piece 39 may be formed back and forth with a rectangular hanging hole 38. A hanging protrusion 34 inserted into the hooked hole 38 is formed at side surface of the lower cover 31 of the position corresponding to the hanging hole (38).

Therefore, when the upper cover 36 is combined to the lower cover 31, the elastic piece 39 become open outward of the upper cover 36 by the hang protrusion 34 and when the hanging protrusion 34 is inserted into the hanging hole, the elastic piece 39 is closed to the side surface of the lower cover 31 by resilience.

The plurality of second through holes 37 are formed at the upper surface of the upper surface 36. The second through-hole 37 is formed at the position corresponding to the first through-hole 35. That is, each a plurality of second through-hole is formed at the position facing each plurality of first through-hole 35. The second through-hole 37 has the same diameter, but may have different diameter as well. The second through-hole 37 is formed at the position corresponding to the first through-hole 35.

The plurality of rotating disks 40 are formed between the lower cover 31 and the upper cover 36. In illustrated example, the plurality of rotating disk 40 is a retractably interlocked structure whereas, the each rotating disk 40 can be independently rotated. In addition, a plurality of the rotating disks 40 is installed but one may installed. In addition, the number of the rotating disk 40 may be changed to various numbers unlike illustrating drawings.

In a example of a shown rotating disk, each rotating disk 40 is threaded with adjacent rotating disk and includes a pinion 41 having diameter the first and second through hole, a first disk portion 43 (see FIG. 4) formed at the lower portion of the pinion 42 and inserted into a first through-hole 35, a second disk portion formed at the upper portion of the pinion 41 inserted into second through-hole 37 and mounted with the implant member 3 and a fastening mean formed at the second disk portion and preventing fluctuation of the implant member when the rotating disk 40 is rotated.

In detail, the fastening means can be applied to a variety of structures and in an embodiment, a fixed rod 47 protruding from the center of the upper surface is applicable. The fixed rod 47 vertically extends at predetermined height from the upper surface of the second disk portion 45. A diameter of fixed rod 47 may be applied in a variety of sizes and had an polygon sectional surface such as a square, triangle, pentangular and the like.

The fixed rod 47 is inserted into a hole formed at the implant member 3 and prevents a shaking or falling of the implant member 3 when the rotating disk 40 is rotated. As shown in FIG. 4, when one example of the implant member 3 is applied to the fixture, the fixed rod 47 may be inserted into a screw hole formed at the inner portion of the fixture.

Meanwhile, the rotating means for rotating the rotating disk 40 is attached to the inner portion of the bases portion 25. In detail, the rotating means includes an electric motor 61 that a driving shaft 61 extends into the inner portion of the body 10 and a driving gear 66 received retractably into the receiving space 33 of the lower cover 31 and combined with the driving shaft 61. The electric motor can rotate the rotating disk 40 from a minimum of 10 rpm to a maximum of 60 rpm. The electric motor 60 may be a DC geared motor. Even if DC geared motor is loaded, there is an advantage that an operation makes possible based on a powerful force at initial operation and makes constant speed control available.

The driving gear 66 is rottenly supported to the lower portion and the upper cover 31, 36. Each supporting shaft 67 protrudes toward the lower portion and the upper portion of the driving gear 66 and each supporting shaft may be inserted into a shaft inserting hole 68, 69 forming at the lower and the upper cover 31, 36. The shaft inserting holes 68, 69 may be formed slightly larger than a diameter of the supporting shaft 67. The combined groove (not shown) of a semi-circular cross-section combined with a driving shaft 61 of the electric motor 66 may be formed at center of the supporting shaft formed at the lower portion of the driving gear 66. In addition, the driving gear 66 rottenly engages with any one of the rotating disks 40. Therefore, when the electric motor 60 is rotated, the driving gear 66 is rotated. According to the driving gear 66 is rotated, the rotating disk 40 is simultaneously rotated.

Meanwhile, a light source array module 50 is attached to a module mounting space (not shown) and is supported by the supporting frame 62. In addition, the module mounting space is shielded by a grille cover 63 combined to a rear surface side of the body 10. The grille cover 63 is formed with a plurality of vent port 65 such that air flows. A plurality of light source inserting holes 15 for inserting the light source attached to the light source inserting holes 15 is formed at the rear surface of the body 10.

The embodiment shows two light source array modules, wherein one of two light source array modules 50 irradiates UV light within the processing chamber of the body 10 and another light source array module is installed to obliquely irradiate UV light from an upper direction to a lower direction within the processing of the chamber 11. This can provide an irradiation of a uniform ultraviolet light on the surface of the implant member 3.

In detail, each light source array module 50 is positioned such that a plurality of light emitting diode chip is spaced apart from one another. The heat sink compound is applied between the emitting diode chip 53 and the heat sink to effectively transfer heat of the light emitting diode chip 53 to the heat sink plate 51. The light source applied to the light source array module 50 is a light emitting diode. A light irradiation by the light emitting diode (LED) does not generate a lot of heat and precisely can control a wavelength to keep a wide beam. The light emitting diode according to the present invention may be LED element irradiating the UV light having a wavelength of 365 nm.

Meanwhile, a control section 70 for controlling an operation of the surface processing device according to an embodiment of the present invention may be a microcontroller. The control section 70 sets an output of the UV light and a rotating speed of the motor and operating time, which user wants, through an operation of an input section 27 and the set details displays a display section 29.

In addition, when user inadvertently opens the door 20 during light irradiation, the user must rapidly turn off the power to ensure a safety to reduce a damage caused by the UV light. In order to achieve this, a sensor section should be installed for sensing an open and close of the door 20 and when the door 20 opens through detecting signal output from the sensor section, the control section 70 is designed to block the power applied to the light source array module 50. At the same time, a buzzer 75 disposed at base portion 25 may generate a beep. In addition, the sensor section may be a limit switch 80.

The limit switch 80 may be installed at a switching mounting section 17 formed at front surface of the body 10 as one example. In addition, when the door 20 is closed, the connecting bar (not shown) formed at the inner side of the door 20 is inserted into the switching mounting section 17 to contact with the limit switch 80.

A method for reforming a dental implant surface using the surface processing device will be described with reference to FIGS. 2 to 5.

The method for reforming the dental implant surface according to the present invention mounts the implant member 3 formed with titanium oxide film the table 30 installed at the processing chamber 11 provided at the body of the surface processing device. At this time, the implant member 3 is attached to only a portion of the rotating disk of the plurality of the rotating disk 40 or the all implant members are attached to each rotating member.

The implant member 3 is attached to the table 30 and then the door 20 is closed and operates the input section 27 to drive the electric motor 60. At this time, a rotating speed of the electric motor may be optionally controlled within 10 to 60 rpm by the operation of the input section 27. The rotating disk 40 is rotated depending on the electric motor 60 and the implant member 3 attached to the rotating disk 40 is rotated together.

At the same time, a power is applied to the light source array module 50, so that UV light is irradiated on the surface of the implant member 3. User can set an output of the UV light and an operating time through the input section 27. The set value displays through the display section 29. In an irradiation process of the UV light, the UV light may be irradiated to the implant member 3 at 2 mW/cm2 of a light amount for 3 hours to 24 hours. The hydrophilic property and the syphilis effectively increase within the light amount and the irradiation time.

### Experimental Example

In order to examine an improved effect of an implant member using the surface treatment device according to an embodiment of the present invention, toxicity and the proliferation for hydrophilic property and syphilis was examined.

1. A Manufacturing of Sample Piece

A disk type sample piece processed by a resorbable blast media (RBM) was manufactured using the same process such as the exiting implant member.

The used sample piece machined titanium (CP Ti Gr4, DYNAMET A Carpenter Company, USA) in the form of a disk of 14 mm of a diameter and 3 mm of thickness within an implant dedicated precision CNC (CINCOM, L20VIII, Japan) and then put the disk a blustering machine and sprayed hydroxide apatite on the surface of the disk and performed RBM process on the disk and then removed the residue from the disk by acid pickling.

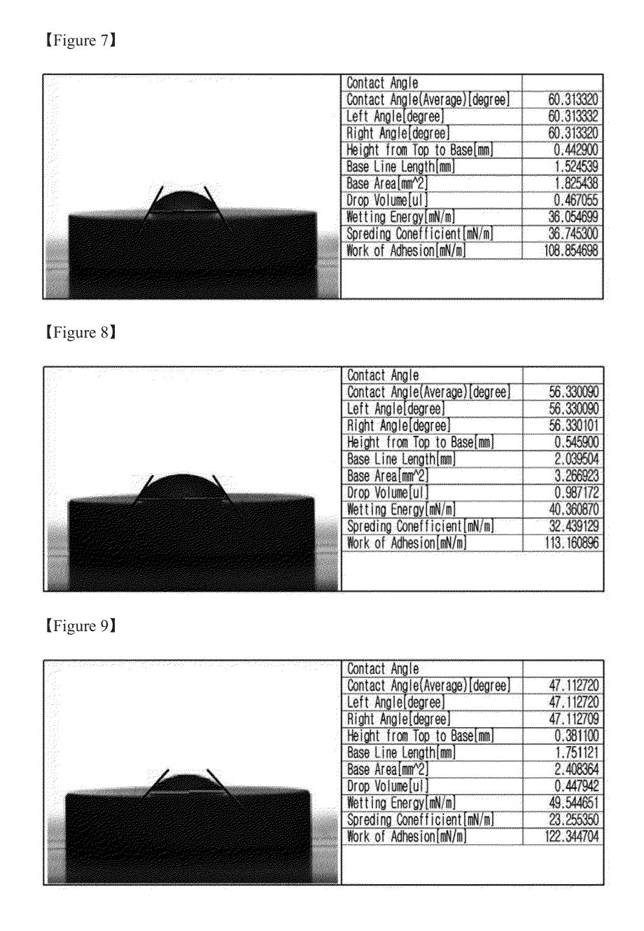

The treated residual of the sample piece analyzed using XPS (X-ray photoelectron Spectrometer)(ESCALAB250, VG Scientific, U.K) and the result was shown in FIG. 6.

Referring to graph of FIG. 6, it was confirmed that the surface-treated disk surface consists of C, O, N, Ti elements.

It was meant that hydroxide apatite (main component: Ca, P) is completely removed by the pickling process. In view of a binding Energy on a data peak, it can be seen that Ti-2P phase and O-1S phase form titanium oxide about each 457 eV, 531 eV. Titanium oxide (TiO2) is a stable oxide and is formed over the entire surface of the disk.

2. UV Light Irradiation

The UV light was irradiated the surface of sample piece using the surface treatment device shown in FIG. 2. At this time, the sample piece was placed on the rotating disk in which the hanging rod is not formed and the UV light was irradiated on the sample piece. The light emitting diode is used as UV light source used UV LED of Nichia Chemical Industry (model: NCSU033A, 365 nm).

The experiment was made that an irradiation time is changed to 2 mW/cm2 of the light amount while rotating the sample piece at about 35 rpm.

3. Hydrophilic Measurement

The hydrophilic measurement for the sample piece used a sessile drop method.

Distilled water drop to each sample of 0.025 ml, a picture was taken against the distilled water dropped sample using a measurement equipment having a video camera (Camscope®, Sometech, Korea) and a contact angle of a left and right in the picture was measured and the measured averaged value was obtained using Dropsnake® (National institute of health, USA).

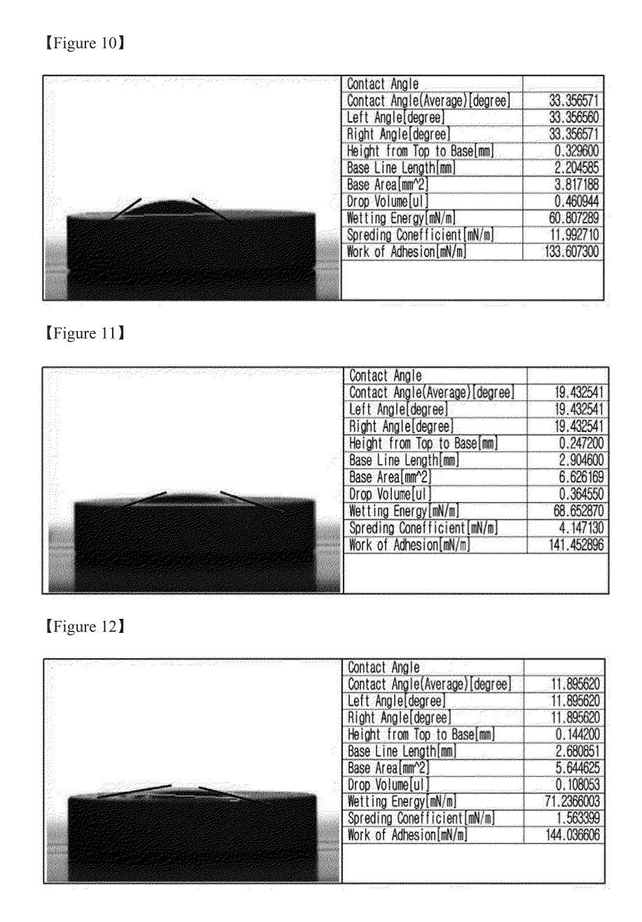

FIGS. 7 to 12 illustrates a picture showing a droplet shape and a average contact angle irradiating the control group and the UV light 365 nm, 2 mW/cm2) for 3 h, 6 h, 12 h, 18 h, 24 h.

Referring to FIGS. 7 to 12, longer irradiation time, an average contact angle became smaller. As shown FIG. 7, it was shown that the sample piece having the irradiation time of the UV light, for example 0 h, that is, the sample on which the average contact angle for the UV light is not irradiated is 60°. In contrast, in sample piece irradiating UK light (hereinafter, referred to as a test group), it was shown that the average contact angle is less than 60°. In a sample piece surface irradiating UK light during 18 hours, the average contact angle was shown 19.43° and thus hydrophilic property appeared to be significantly increased. This was shown that the reforming result of a hydrophilic property for the implant surface improves by Ti—OH induced by irradiating the UK light on titanium oxide film.

4. Cell Toxicity and Cell Proliferation Experiments.

The toxicity and proliferation for osteoblast was confirmed whether a surrounding cell has the toxicity when UV light irradiated implant member is implanted into the body.

The used osteoblast in the experiment used MG63 (osteosarcoma, primary, human, Korea cell line Bank) cell. In order to culture cells, DMEM (minimal essential medium)(Gibco, Grand Island, N.Y., USA) solution containing 10% fetal bovine serum (FBS, Gibco, Grand Island, N.Y., USA), penicillin of 100 U/ml and streptomycin of 100 μg/ml put into a cell culturing plate of size of diameter of 100 mm and cultured this under the condition of 37° C., humidity 100% and 5% CO2 in a culture medium. When cell become 70% confluent state, they performs a subculture.

The samples irradiating the UK light (365 nm, 2 mW/cm2) for 6 h, 12 h, 18 h, 24 h put into a culturing plate (corning Co., USA) of 24 Well one by one, poured the cell culturing solution by 5×104 into the plate, added 10% DMEM of 400 μl to well and processed this in 6 hours, cultured this in the cell culture medium for 1, 4 and 7 days and confirmed the toxicity and a growth aspects through XTT assay.

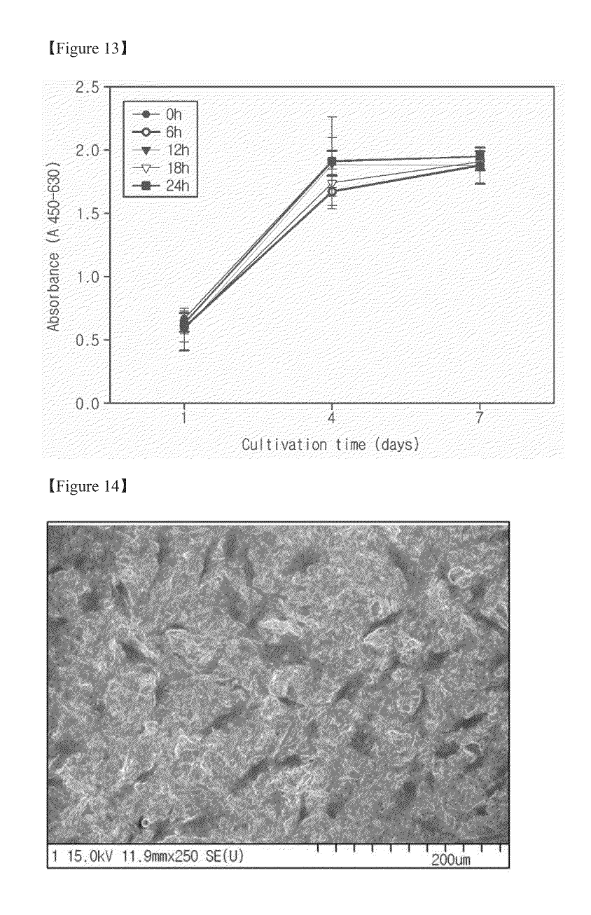

The absorbance of the cell culturing culture fluid of 200 μl was measured at A450 and the absorbance value for the culture fluid and reagents was measured at A630 and corrected a value of the A450. The experimental results are shown in tables 1 to 3 and a grapy of FIG. 13.

TABLE 1

| | UV irradiation | | | | |
|---|---|---|---|---|---|
| Sample | 0 | 6 | 12 | 18 | 24 |
| A450 | 0.716 | 0.781 | 0.832 | 0.794 | 0.802 |
| | 0.714 | 0.550 | 0.617 | 0.633 | 0.610 |
| | 0.760 | 0.663 | 0.500 | 0.690 | 0.696 |
| A630 | 0.067 | 0.068 | 0.069 | 0.072 | 0.071 |
| | 0.061 | 0.06 | 0.064 | 0.064 | 0.062 |
| | 0.065 | 0.07 | 0.066 | 0.067 | 0.067 |
| A450-630 | 0.649 | 0.713 | 0.763 | 0.722 | 0.731 |
| | 0.653 | 0.490 | 0.553 | 0.569 | 0.548 |
| | 0.695 | 0.593 | 0.434 | 0.623 | 0.629 |
| Average | 0.666 | 0.599 | 0.583 | 0.638 | 0.636 |
| Deviation | 0.025 | 0.112 | 0.167 | 0.078 | 0.092 |
| 100% | 100 | 89.9 | 87.5 | 95.7 | 95.4 |
| conversion (%) | | | | | |

As known in the result of the table 1, the absorbance in one day of the culture shows somewhat lower than the control group if the UK light is irradiated for 6 hours and 12 hours, but shows no greater difference than the control group if the UK light is irradiated for 18 hours.

TABLE 2

| | UV irradiation | | | | |
|---|---|---|---|---|---|
| Sample | 0 | 6 | 12 | 18 | 24 |
| A450 | 1.874 | 1.864 | 1.868 | 1.931 | 1.894 |
| | 1.691 | 1.763 | 1.954 | 1.712 | 2.191 |
| | 2.374 | 1.599 | 2.069 | 1.803 | 1.83 |
| A630 | 0.07 | 0.068 | 0.072 | 0.072 | 0.069 |
| | 0.066 | 0.07 | 0.07 | 0.071 | 0.066 |
| | 0.07 | 0.074 | 0.073 | 0.073 | 0.077 |
| A450-630 | 1.804 | 1.796 | 1.796 | 1.859 | 1.825 |
| | 1.625 | 1.693 | 1.884 | 1.641 | 2.125 |
| | 2.304 | 1.525 | 1.996 | 1.730 | 1.753 |
| Average | 1.911 | 1.671 | 1.892 | 1.743 | 1.901 |
| Deviation | 0.352 | 0.137 | 0.100 | 0.110 | 0.197 |
| 100% | 100 | 87.4 | 99.0 | 91.2 | 99.4 |
| conversion (%) | | | | | |

As known in the result of the table, the absorbance in 4 days shows approximately 13% lower than the control group if the UV light is irradiated for 6 hours, but shows no greater difference than the control group if the UV light is irradiated for more than 12 hours.

TABLE 3

| | UV irradiation | | | | |
|---|---|---|---|---|---|
| Sample | 0 | 6 | 12 | 18 | 24 |
| A450 | 1.948 | 1.974 | 2.077 | 2.014 | 1.995 |
| | 2.047 | 1.89 | 1.932 | 1.911 | 2.058 |
| | 1.928 | 1.948 | 1.796 | 1.996 | 1.985 |
| A630 | 0.065 | 0.059 | 0.06 | 0.063 | 0.064 |
| | 0.066 | 0.062 | 0.063 | 0.063 | 0.064 |
| | 0.073 | 0.067 | 0.068 | 0.068 | 0.071 |
| A450-630 | 1.883 | 1.915 | 2.017 | 1.951 | 1.931 |
| | 1.981 | 1.828 | 1.869 | 1.848 | 1.994 |
| | 1.855 | 1.881 | 1.728 | 1.928 | 1.914 |
| Average | 1.906 | 1.875 | 1.871 | 1.909 | 1.946 |
| Deviation | 0.066 | 0.044 | 0.145 | 0.054 | 0.042 |
| 100% | 100 | 98.3 | 98.1 | 100.1 | 102.0 |
| conversion (%) | | | | | |

As known in the result of the table 3, the absorbance in 7 days of the culture shows no greater than the control group.

In view of the result of tables 1 to 3 and FIG. 13, the test group shows no difference in the cell toxicity and proliferation compared with the control group. A cell survival rate is shown 95.3% (a numerical integration of all values) compared with the control group to 7 days of the cell culture and the cell survival rate of about less than 87% is not shown at measurement of the number of the cells for 1, 4 and 7 days of each sample. From the above results, it was confirmed what irradiates the UV light on the implant member do not have a significant effect on the cell toxicity and proliferation.

5. Cell Attachment Test

It was conformed that the osteoblast is attached to the sample piece so that a material has an effect on an attachment reaction of the surrounding cells when the UV light irradiated implant member is implanted into the body. For the test, the sample pieces irradiating the UV light for 6 h, 12 h, 18 h and 24 h put into the culturing dish (corning Co., USA) of 24 Well, the culture fluid of osteoblast poured by 5×104 and then added 10% DMEM 400 μl to each well and processed this. In addition, the culture fluid is removed in 6 hours, the cells are pretreated for observation of the scanning electron microscopy and confirmed cell attachment.

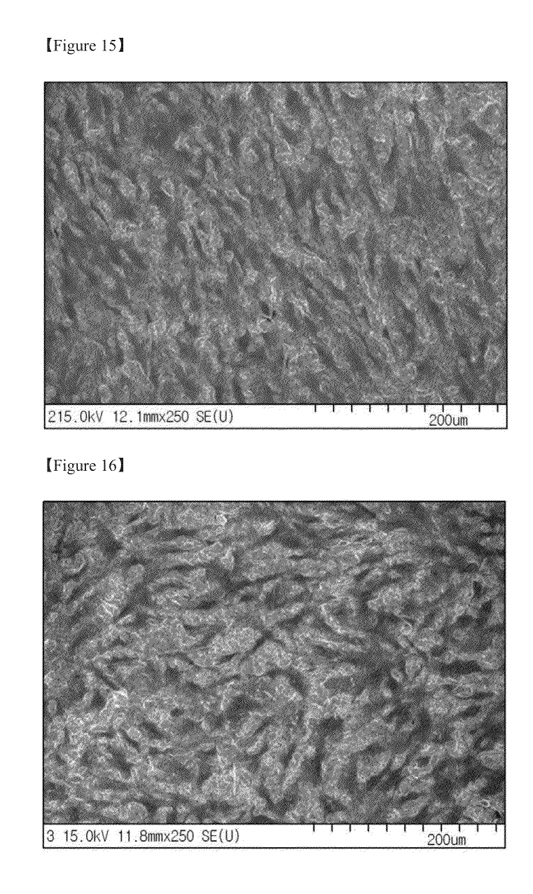

In FIGS. 14 to 18, the control group and the image of osteoblast attached to the sample piece irradiating UV for 6, 12, 18 and 24 hours is a 250 time magnified picture. In addition, the cell numbers of an image showing the table 4 and FIG. 14 to FIG. 18 are checked and the number of the attached cells is quantified. A quantitative level of cell attachment images five areas (up, down, left, right and middle).

TABLE 4

| | | UV irradiation | | | | |
|---|---|---|---|---|---|---|
| Sample | | 0 | 6 | 12 | 18 | 24 |
| Attachment | up | 88 | 120 | 125 | 111 | 99 |
| cell | down | 131 | 125 | 131 | 125 | 122 |
| | left | 102 | 119 | 104 | 111 | 135 |
| | right | 110 | 125 | 111 | 102 | 122 |
| | middle | 84 | 75 | 95 | 100 | 88 |

| | | | | | |
|---|---|---|---|---|---|
| Average | 103 | 112.8 | 113.2 | 109.8 | 113.2 |
| Deviation | 18.8 | 21.3 | 14.8 | 9.9 | 19.1 |
| 100% | 100.0 | 109.5 | 109.9 | 106.6 | 109.9 |
| conversion (%) | | | | | |

As a while, the number of the test group was observed higher than the number of the cell attached to the control (an average 108.9% of the attachment rate compared to the control group).

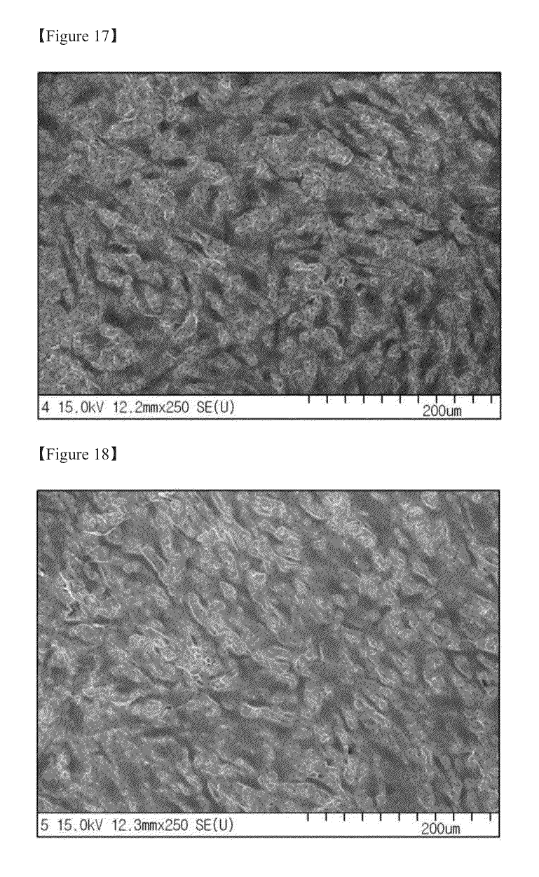

In FIGS. 19 to 23, the control group and the image of osteoblast attached to the sample piece irradiating UV for 6, 12, 18 and 24 hours is a 1000 time magnified picture. As a result of 1,000 magnifications image observations that a cell proliferation and a cell-cell contact identified in the differentiation process was confirmed in the sample piece searching the UV light.

To summarize the above result, it can be seen that in the test group, the hydrophilic property reduces the average contact angle of 60° to 19.43° or less to improve the hydrophilic property and do not have a significant effect on the cell toxicity and proliferation. In addition, the test group for attachment reaction with the surrounding cells at implant into the body is reported that it has more member of cell attachment than the control group and cell.

Therefore, it can be seen that the present invention provides the hydrophilic property to an implant member surface by irradiating an ultraviolet to naturally surface of implant member coated with a formed titanium oxide film or an artificially formed titanium oxide film. It is likely that the surface-reformed implant member with the hydrophilic property promotes an interaction with a biological solution, cell and tissues to improve an osseointegration.

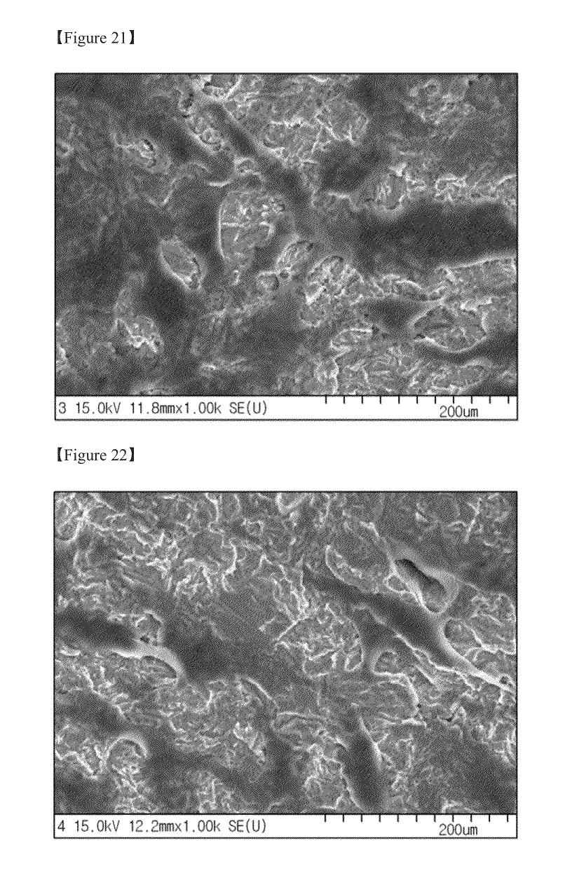

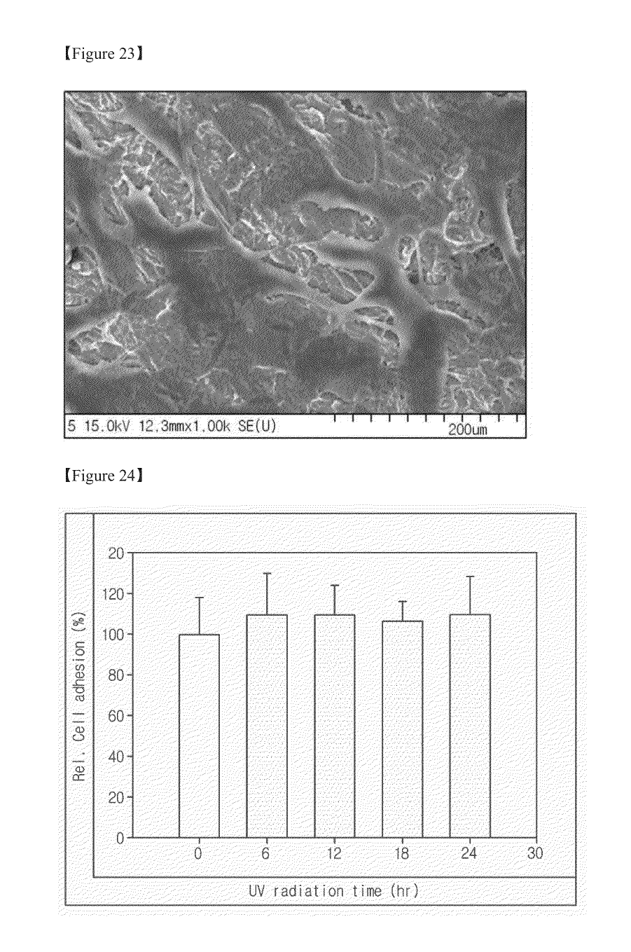

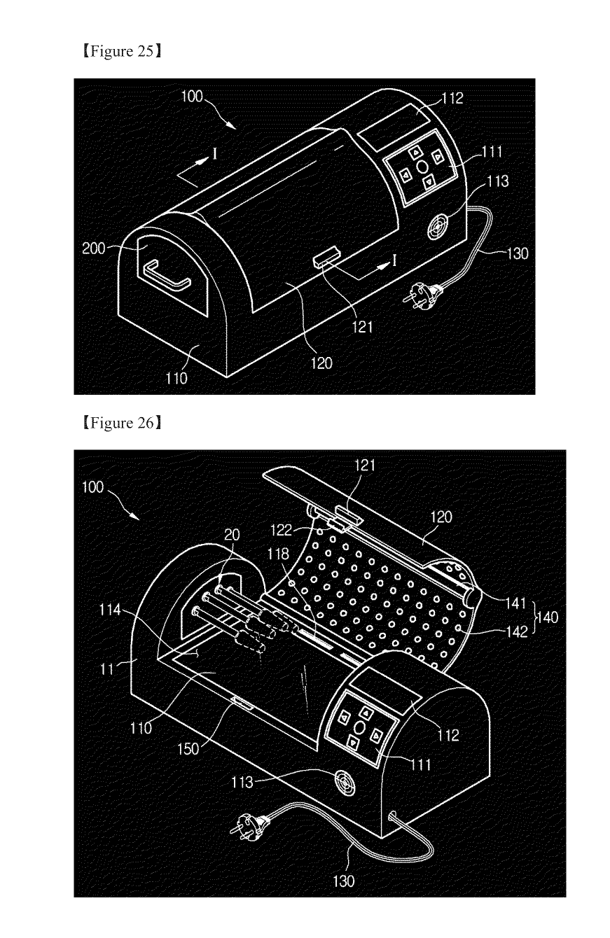

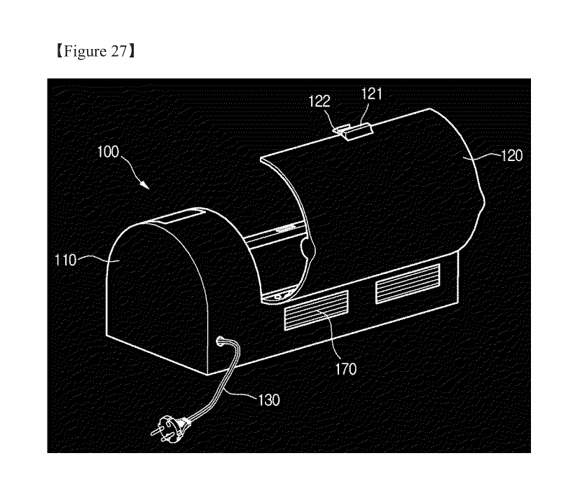

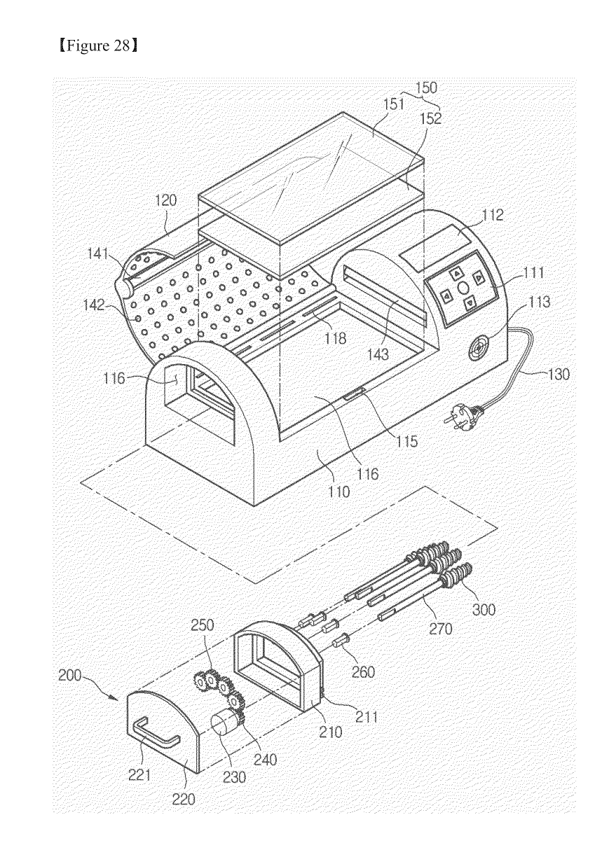

FIG. 25 is a front perspective view of a dental surface treatment device according to a second embodiment of the present invention. FIG. 26 is a front perspective view showing a opened cover of the surface treatment processing device. FIG. 27 is a rear perspective view showing a opened cover of the surface treatment processing device. FIG. 28 is a exploded perspective view of the surface treatment device and FIG. 29 is a longitudinal cross-sectional view taken along FIG. 25.

Referring to FIGS. 25 to 29, the surface treatment processing device 100 of the dental implant according to a second embodiment of the present invention includes a body 100 provided with a treatment chamber 114 for treating the surface, a cover covering an upper side of opening portion of the treatment chamber 114 and a mounting module 200 slidably inserting and withdrawing from one side of the body at state mounted with the surface processing object. In addition, a power cord 130 extends from the rear surface of the body 110 and the power supply source supplied through the power cord 130 is divided and supplied to a driving motor of the mounting module and cooling pan, a light source and a control panel as described below.

In detail, the treatment chamber 114 is recessed or is formed to have a step from the upper surface of the body 110 in predetermined depth. In addition, the treatment chamber 114 may be selectively opened and closed by the treatment cover 120. That is, the treatment chamber 114 forms a single closed space by closing the cover 120.

A tray seating groove 116 is formed at the bottom of the treatment chamber 114 and the tray is seated at the tray seating groove 116. The surface treatment, for example fixture may be placed at the tray 150 at upstanding state. In detail, a head portion of the fixture becomes upright in contact with the tray 150.

In addition, the tray 150 includes a reflective plate 152 placed in the seating groove 116 and a transparent plate 151 placed in an upper surface of the reflective plate 152. The reflective plate 152 is placed at a lower side of the transparent plate 151, so that the light irradiating from a light source (described later) pass through the transparent plant 151 and then reflects on off the reflective plate 152. In addition, a portion of light reflected from the reflective plate 152 may irradiated to the bottom of the fixture. For example, when the light source is mounted on the upper side of the tray 150, a portion of the surface of the fixture does not receive the light irradiating from the light source and is likely to evenly the surface treatment. To avoid this, the fixture is placed at the transparent 152 and the light reflected from the reflective plate 152 placed at a lower side of the transparent plate 151 is irradiated to the bottom of the fixture. To that end, the transparent plate 151 and the reflective plate 152 is preferably placed in a determined interval. That is, the surface treatment object is spaced apart from the reflective plate 152, so that the light reflected from the reflective plate 152 is irradiated to the surface treatment object.

In addition, a slit shaped intake port 118 may be formed at one side of an edge of the bottom portion of the treatment chamber 114. In detail, when the light source is ON to treat the surface, because an internal portion of the treatment chamber 114 becomes hot, the internal portion of the treatment chamber 114 should be cooled. To this end, the intake port 118 is formed at an inner periphery surface of the treatment chamber 114 and an air flow structure discharging air into the outside through any one surface of the body 110 is provided.

In more detail, in order to form the air flow structure, as shown in FIG. 29, the cooling pan 160 is placed in the inner portion of the body 110, specifically, the inner portion of the body corresponding to a lower side of the bottom portion of the treatment chamber 114 and a exhaust grillee 117 is formed at one surface of the body 110. As an embodiment, the intake port 118 is formed at the edge of the rear side of the bottom and the exhaust grillee 117 is formed at the rear surface of the body 110. In addition, the intake port of the cooling pan 160 is in communication with the intake port 118 and the exhaust port 117 is in communication with the exhaust grillee 117. The cooling pan 160 is possible even any kind of fan and in particular, may be selected from a transverse flow fan, a centrifugal fan and an axial fan. In the embodiment, a sirocco fan such as the transverse pan may be applied.

Meanwhile, the lower surface of the cover 120 is mounted with the light source 140 emitting the UV light. In detail, the light source 140 includes a halogen lamp, a high pressure mercury UV lamp, a light emitting diode (LED) or an organic light emitting diode, which reach from a left end portion to a right end portion of the cover 120.

In more detail, the light source 140 may include a first light source 141 containing the halogen lamp or the high pressure mercury UV lamp or a second light source containing the light emitting diode or the organic light emitting diode. In present embodiment, the first 141 is mounted across the center of the bottom surface of the cover 120 and the second light source 142 is in plurality mounted at the bottom of the cover 120 except for an area of the first light source 141, but is limited to thereto. That is, only one of the first light 141 and the second light source 142 may be mounted at the cover 120. In addition, a third light source 143 may further be mounted at side surface portion of the treatment chamber 114. The third light source 143 is applicable to any one or all of the above-mentioned kinds of the light source. In addition, the light source 140 emits the UV light having a wavelength of 300˜400 nm and preferably emits the UV light having a wavelength of 365 nm.

Moreover, the cover 120 is formed in a round type, so that light reflected from the reflective plate 152 impacts on the lower surface. Therefore, the UV light is evenly irradiated to the surface of the surface treatment object placed within the treatment chamber 14 to increase a uniformity of the surface treatment. In addition, a locking member 121 protrudes from the front surface portion of the cover 120, easily lift the cover 120, with the user holding the locking member with finger and prevent the cover to naturally be opened or shacked. In detail, the locking member 121 includes the protrusion 121 protruding from the front surface portion of the cover 120 and a hook 122 from a front border of the cover 120. The hook 122 may be formed integrally with the protrusion 121. In addition, the front edge of the treatment chamber 114 is formed with a slot 115 into the hook 122 is inserted. In addition, with the hook 122 being inserted the slit 115, the hook 122 may be a structure in which is separated from the slots 115 by pressing the protrusion 121. In addition, the edge of the rear side of the cover 120 is ratably connected to the rear side, especially the edge of the rear side of the treatment chamber 114. Therefore, in particularly, if a user lifts the protrusion 121 while pressing it, the cover 120 is upwardly rotated to be opened.

In addition, the cooling member may be mounted within the cover 120. That is, the cooling member in place of the cooling pan may be mounted and the cooling member may be mounted together with the cooling pan 160. In addition, when a power is supplied, one side surface thereof is cooled and an opposite side may include a thermoelectric element forming a heat sink emitting a heat. The thermoelectric element may include a Negative Temperature Coefficient thermistor using Peltier effect or a positive Temperature Coefficient thermistor. The thermoelectric element may be a rectangular flexible plate and may close depending on a shape of the cover 120 within the cover 120.

On the other hand, a module hole 116 into which the mounting module 200 is inserted is formed at side surface portion of the body 110 opposite to a side surface mounted with the third light source 143.

In detail, the mounting module 200 includes a module case 210, a case cover 220 covering a opening surface of the module case 210, a handle 221 formed an outer periphery surface of the case cover 220 and an driving mechanism received in the inner portion of the module case 210. The drive mechanism includes a driving motor 230 providing a torque, a driving gear 240 connected to an output shaft of the driving motor 230 and a plurality of driven gear 250 engaging with the driving gear 240. In addition, the surface processing object such as the fixture is connected to center of the plurality of driven gear 250.

In more detail, one end of each drill shaft 270 is inserted into the center of the plurality of driven gear 250. The connector 260 is inserted into the center of the driven gears 250 and the connector 260 protrudes from a inner surface of the module case 210, that is, the opposite surface to surface mounted with the case cover 220. In addition, a one end of the drill shaft 270 is inserted into the connector 260. In present embodiment, if the surface processing object including fixture 300 is combined with other end of the drill shaft 270, the one end of the drill shaft 270 is inserted into each one of plurality of driven gears 250 through the connector 260, but is limited to thereto. That is, the method for connecting the fixture 300 and connecting the fixture to the connector 260 is possible using a mounting means. What the drill shaft 270 is described as one example of the mounting means is why a hand drill is used to implant the fixture 300 into a drill groove firmed in an alveolar periosteum in the process of the implant surgery. That is, this is why the surface is treated at condition inserting the fixture 300 into an end portion of the drill shaft inserted into the head drill and connects the drill shaft to a body of the handle drill without the need to separate the surface treated fixture 300 and then the fixture into implant an alveolar periosteum. So, when user holds the fixture, a bacterium is not delivered to the fixture 300.

In addition, a terminal 211 is protruded from the module case 210 and a socket for inserting the terminal may be formed at one surface of the module hole 116 corresponding to the terminal. Therefore, when the mounting module 200 is inserted into the module hole 116, the terminal 211 is inserted into the socket. Therefore, the power supplied through the power cord 130 is supplied by the driving motor 230. In addition, the mounting module 200 is separated from the module hole 116 and simultaneously the power supply to the driving motor 230 is cut off. Further, it is preferable that a control circuit is designed such that the mounting module 200 is separated and simultaneously the power supply to the light source 140 is cut off. This can prevents the phenomenon that a portion of the UV irradiated from the light source 140 is leaked through the module hole 116 to the outside.

The body 100 is provided with an input section 111 for setting and adjusting various commands and a display section 112 capable of checking the inputted commend through the input section 111 and an operating status of the surface treatment device 100. The various commands include an irradiation time and wavelength of the light source 140, a rotational speed (air flow) of the cooling fan 150. A PCB circuit board is connected to the rear surface of the input section 111 and the display section 112, and they can be defined as a control panel or a controller.

A speaker 113 may be mounted at the front surface of the body 110. In detail, when the inputted irradiation time is reached, the cover 120 is in advertently opened or the mounting module 200 is separated from the body 100, or the inner portion of the treatment chamber 114 is overheated, a beep is output through the speaker 113.

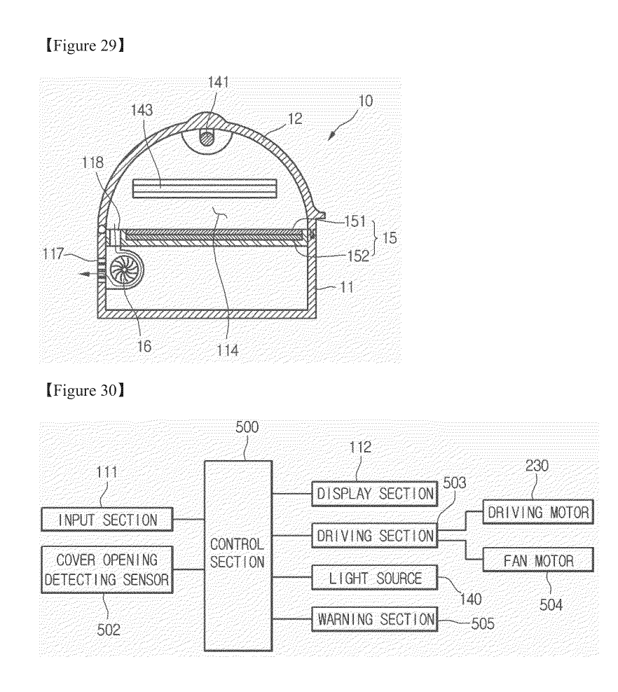

FIG. 30 is a block diagram schematically showing a control system including the surface treatment device of the dental implant according to a second embodiment of the present invention.

Referring FIG. 30, the surface treatment device 1100 according to an embodiment of the present invention includes control section 500, an input section 111 for inputting various command to the control section 500, a display section 112 representing a inputted command content and/or the operating condition of the surface treatment device 100, a light source 140 for irradiating the UV light, a driving section 503 including the driving motor 230 and a fan motor 504 driving the cooling fan 160, a warning section 505 for outing a warning sound and a cover opening detecting sensor 502 for sensing a opening the cover 120.

In detail, the warning section 505 may include the speaker 113. In addition, the cover opening detecting sensor 502 may be mounted at an area contacting the cover 120 with the body 110. Alternatively, the hook 122 of the cover 120 may be mounted at inner portion of the slot inserting the hook 122. That is, when the hook 122 is removed from the slot, the cover opening detecting sensor 502 is designed to sense this. In addition, when the cover opening is sensed by the cover opening detecting sensor 502, the power supply to the light source 120 is cut off, so that the irradiation of the UV light may be programmed to be stopped.

According to a control system including the above-mentioned configuration, it can input an light irradiation time, a rotational speed of the driving motor 230 and an air flow of a cooling fan 160. In addition, the inputted air flow is transferred to the control section 500 and an electrical signal corresponding to the command inputted from the control section. The command information inputted therewith is controlled in a control section to be output through the display section 112. In addition, the light irradiation corresponding to the inputted command is set and a time counter of the timer (not shown) is started.

Meanwhile, various types of audio information may be programmed to be output through the warning section 505. For example, if the command input is finished and press a start button, the audio information“A surface treatment is finished” is output and if the surface treatment is finished, the audio information “A surface treatment is finished” is output. The audio information “Open the cover and take out the object” may be output. In addition, if an operation for opening the cover 120 during the surface treatment, an Auto-Lock for block an opening of the cover is set and simultaneously the warning massage “You cannot currently open the cover because it is in an irradiation process of a UV light.” may be output. In addition, if the mounting module 200 is fetched from the body 110, the irradiation of the UV light from the light source 140 is stop and simultaneously the audio massage “Stop an irradiation because a mounting module is separated.” may be output.

In this case, the cooling fan 160 may be programmed to be stopped and the operation is programmed to be kept during a setting time from the stop time of the UV light irradiation and then to be stopped.

As such, the hydrophilic property is improved by irradiating the UV light to the implant fixture 300 to provide improved syphilis. A time of the implant member exposed to air before a procedure is minimized by implanting into a patient just if the irradiation of the UV light is start just before procedure and the irradiation of the UV light is finished.

In addition, Since the UV light is irradiated under a condition that inserts the object to be treated, that is, the fixture into the drill shaft of the hand drill; user directly does not touch or hold the fixture by hand, thereby minimizing a risk of infection.

1. A surface treatment device of dental implant, comprising:
a body providing a treatment chamber to receive a surface processing object;
a cover combined to the body to selectively open the treatment chamber;
a light source irradiating UV light to a surface of the surface treatment object; and
a reflective plate positioned at a bottom of the treatment chamber to reflect the UV light emitting from the light source toward the surface treatment object.

2. The surface treatment device of claim 1, further comprising a mounting module detachably combined to the body and inserting the surface treatment object to an inner portion the treatment chamber in a status where the surface treatment object is coupled to the mounting module.

3. The surface treatment device of claim 2, wherein the mounting module includes a driving mechanism to rotate the surface treatment object.

4. The surface treatment device of claim 3 wherein the mounting module includes:
a module case receiving the driving mechanism;
a handle formed at an outer periphery surface of the module case; and
a terminal projected to one surface of the module case and supplying power to the driving mechanism.

5. The surface treatment device of claim 4, wherein the driving mechanism includes:
a drive motor generating a torque by the power supplied through the terminal;
a drive gear connected to the drive motor; and
one or more driven gears engaged to the drive gear.

6. The surface treatment device of claim 5, wherein the driving mechanism further includes:
a connector inserted into a center portion of the driven gear; and
a shaft member having one end separably inserted into the connector and other end separably connected to the surface treatment object.

7. The surface treatment device of claim 2, wherein one side of the body is formed with a receiving portion receiving the mounting module, and the mounting module is configured to slide to be inserted to or retrieved from the receiving portion.

8. The surface treatment device of claim 2, wherein when the mounting module is separated from the body, a supply of power to the light source is blocked.

9. The surface treatment device of claim 1, further comprising a cooling fan provided in an inner portion of the body and cooling an inner portion of the treatment chamber.

10. The surface treatment device of claim 9, further comprising:
an intake port formed in one side of the bottom portion of the treatment chamber to absorb the air inside the treatment chamber; and
an exhaust grille formed in one surface of the body to discharge the air inside the treatment chamber to an outside.

11. The surface treatment device of claim 10, wherein an intake port of the cooling fan is in communication with the intake port of the bottom of the treatment chamber, and a discharging port of the cooling fan is in communication with the exhaust grille of the body.

12. The surface treatment device of claim 1, further comprising a thermoelectric element mounted on an inner portion of the cover and cooling the treatment chamber.

13. The surface treatment device of claim 1, wherein the light source is mounted on an inner side surface of the cover, and includes any one or both a first light source containing at least a halogen lamp or an high pressure mercury UV lamp and a second light source containing a light emitting diode or an organic light emitting diode.

14. The surface treatment device of claim 13, further comprising a third light source mounted on one side of an inner periphery surface of the treatment chamber,
wherein the third light source is the same kind of light source as any one of the first light source or the second light source.

15. The surface treatment device of claim 1, wherein the cover is curved in predetermined curvature and is rotatably coupled to the body.

16. The surface treatment device of claim 1, further comprising a transparent plate placed above the reflective plate.

17. The surface treatment device of claim 16, wherein the transparent plate is placed upwardly spaced from the reflective plate.

18. The surface treatment device of claim 1, further comprising a sensor detecting an opening of the cover,
wherein when the opening of the cover is detected by the sensor, a supply of power to the light source is blocked.

19. The surface treatment device of claim 1, further comprising a control panel formed at any one side of the body,
wherein the control panel includes:
an input section for inputting various commands; and
a display section showing at least one of a command information inputted through the input section and an operating condition of the surface treatment device.

20. The surface treatment device of claim 1, further comprising a warning section informing at least one of a start and finishing time of the surface treatment process, a malfunction, an overheat within the treatment chamber and an abrupt condition.

21. The surface treatment device of claim 1, wherein the light source emits a UV light having 365 nm of wavelength.
